(19) United States
(12) Patent Application Publication
(10) Pub. No.: US 2008/0145352 A1
(43) Pub. Date: Jun. 19, 2008

(54) Method for promoting axonal re-growth and behavior recovery in spinal cord injury

(75) Inventors: Wen-Cheng Huang, Taipei (TW); Wen-Chun Kuo, Taipei (TW); Henrich Cheng, Taipei (TW)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: Henrich Cheng

(21) Appl. No.: 11/973,426

(22) Filed: Oct. 9, 2007

Related U.S. Application Data
(60) Provisional application No. 60/828,450, filed on Oct. 6, 2006.

(51) Int. Cl. A61K 38/51; A61P 25/00

(52) U.S. Cl. 424/94.5

(57) ABSTRACT

A method for promoting axon re-growth and behavior recovery in a subject suffering from a nerve injury is described. The method includes administering a pharmaceutical composition comprising a safe and effective amount of a chondroitinase ABC (CHABC) to an injury site of the nerve injury in the subject.

### CROSS-REFERENCE TO RELATED APPLICATIONS

This application claims priority to U.S. Provisional Patent Application No. 60/828,450, filed on Oct. 6, 2006, entitled “A Method For Promoting Axonal Re-Growth And Behavior Recovery In Spinal Cord Injury.”

### BACKGROUND OF THE INVENTION

The present invention relates to a method for promoting axonal re-growth and behavior recovery in spinal cord injury.

In mammals, the adult neural tissues have limited regenerative capability. Therefore, numerous brain or spinal cord injuries are unable to repair themselves, or re-establish functional connections. Many complicated causes, including immunoresponse-molecules and inhibitory factors, interfere with the regenerative capacity of adult central nervous system. Among the inhibitory factors, chondroitin sulphate proteoglycans (CSPGs) are up-regulated via astrocytes and oligodendrocytes in the injury site after spinal cord injury (SCI) and limit axonal regeneration.

Some reports have noted that CSPG expression is generally up-regulated after injuries. Most studies have focused on changes in expression of members of a single family of CSPG after different lesion methods. CSPGs are composed of glycosaminoglycan (GAG), which is a key factor to block axon re-growth after injury. Chondroitinase ABC (ChABC) is a bacterial enzyme, which digests the GAG side chain of CSPGs. However, CHABC is rarely used in clinical trials of spinal cord injury cases due to its toxicity.

There remains a need for new methods and compositions for promoting axonal re-growth and behavior recovery after a spinal cord injury. The present invention satisfies this need.

### BRIEF SUMMARY OF THE INVENTION

The present invention relates to the use of a safe and effective amount of ChABC for treating nerve injury in a subject.

One aspect of the invention relates to a method for promoting axon re-growth and behavior recovery in a subject suffering from a nerve injury, which comprises administering a pharmaceutical composition comprising a safe and effective amount of a chondroitinase ABC (CHABC) to an injury site of the nerve injury in the subject. In embodiments of the present invention, the pharmaceutical composition comprises about 0.1 U/ml to about 10 U/ml, preferably about 0.5 U/ml to about 5 U/ml and more preferably about 1 U/ml, of the ChABC.

Another aspect of the invention relates to a pharmaceutical composition for promoting axon re-growth and behavior recovery in a subject suffering from a nerve injury. The pharmaceutical composition comprises a safe and effective amount of a chondroitinase ABC (ChABC) and a pharmaceutically acceptable carrier.

Additional aspects and advantages of the invention will be set forth in part in the description which follows, and in part will be apparent from the description, or may be learned by practice of the invention in view of the present disclosure. The benefits and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The patent or application file contains at least one drawing executed in color. Copies of this patent or patent application publication with color drawings will be provided by the Office upon request and payment of the necessary fee.

The foregoing summary, as well as the following detailed description of the invention, will be better understood when read in conjunction with the appended drawings, which are shown for the purpose of illustrating the invention. It should be understood, however, that the invention is not limited to the precise arrangements and instrumentalities shown.

In the drawings:

FIG. 1 is a graph illustrating the Basso, Beattie, Bresnahan (BBB) open field locomotor's test scores of female SD rats after T8 transection in different treatment groups according to the present invention;

FIG. 2A shows morphologies of spinal cords 8 weeks after T8 transection in different treatment groups according to the present invention;

FIG. 2B shows histochemical cross-sections of spinal cords 8 weeks after T8 transection in different treatment groups according to the present invention;

FIG. 3 shows histochemical cross-sections of the spinal cord injury region 8 weeks after T8 transection in different treatment groups according to the present invention;

FIG. 4 shows histochemical cross-sections of CS-56 immunostained rat spines in the CHABC 1 U/ml group and control group;

FIG. 5 shows histochemical cross-sections of 2B6 immunostained rat spines in the ChABC 1 U/ml group and the control group;

FIG. 6 shows histochemical cross-section of HRP traced rat spine in the ChABC 1 U/ml group;

FIG. 7A shows histochemical cross-section of GAP-43 immunostained rat spine in the ChABC 1 U/ml group;

FIG. 7B is a high resolution image of the rostral stump lesion site (B) taken from FIG. 7A; and

FIG. 7C is a high resolution image of the central area of the scar tissue (C) taken from FIG. 7A.

### DETAILED DESCRIPTION OF THE INVENTION

Unless defined otherwise, all technical and scientific terms used herein have the same meaning as commonly understood by one of ordinary skill in the art to which the invention pertains. Although any methods and materials similar or equivalent to those described herein can be used in the practice of testing of the present invention, the preferred materials and methods are described herein.

As used herein, the article “a” or “an” means one or more than one (that is, at least one) of the grammatical object of the article, unless otherwise made clear in the specific use of the article in only a singular sense.

For a better understanding of the present invention, some of the terms used herein are explained in more detail. It is also to be understood that the terminology used herein is for the purpose of describing particular embodiments only, and is not intended to be limiting. In describing and claiming the present invention, the following terminology will be used in accordance with the definitions set out below.

As used herein, the term “subject” refers to an animal, who has been the object of treatment, observation or experiment. Examples of a subject include, but are not limited to, any vertebrates suffering from a nerve injury. The vertebrates include, but not limited to, humans, commercially relevant mammals such as cattle, pigs, horses, sheep, cats, dogs, mice, rats, rabbits, and pets, birds including commercially relevant birds such as chickens, ducks, geese, and turkeys.

As used herein, the term “safe and effective amount” means that amount of an active compound or pharmaceutical agent that elicits the biological or medicinal response in a tissue system or in a subject that is being sought by a researcher, veterinarian, medical doctor or other clinician, which includes alleviation of the symptoms of the disease or disorder being treated, without causing toxic side effects to the subject. The administration of a safe and effective amount of ChABC to a subject suffering from a nerve injury results in a clinically observable beneficial effect, i.e., improved axon re-growth and behavior recovery in the subject, without causing observable toxic side effects to the subject. In one embodiment of the invention, the administration of a safe and effective amount of ChABC to a subject suffering from a nerve injury results in axon re-growth and behavior recovery in the subject, so that the nerve injury is about any of 10%, 20%, 30%, 40%, 50%, 60%, 70%, 80%, or 90% of that would have been had the subject not received the safe and effective amount of ChABC.

A useful assay for confirming a safe and effective amount (e.g., a therapeutically effective amount) of ChABC is to measure the degree of recovery from a nerve injury. The safe and effective amount can be estimated using either a cell culture assay or an appropriate animal model. The animal model is used to achieve a desired concentration range and an administration route. Thereafter, such information can be used to determine a dose and route useful for administration into humans. The safe and effective amount actually administered to a subject depends on the subject to be treated and the degree of injury.

The therapeutic effect and toxicity of ChABC may be determined by standard pharmaceutical procedures in cell cultures or experimental animals in view of the present disclosure. The dose ratio between therapeutic and toxic effects is a therapeutic index, and it can be expressed as the ratio of ED₅₀/LD₅₀, wherein ED₅₀ is a dose therapeutically effective for 50% of a population; and LD₅₀ is a dose lethal to 50% of a population. Pharmaceutical compositions which exhibit high therapeutic indices are preferable. The data obtained from cell culture assays and animal studies can be used for formulating a dosage range for use in humans. The dosage of such compounds lies preferably within a range of circulating concentrations that include the ED₅₀, with little or no toxicity. Such a dosage may vary within this range depending upon the dosage form employed, the susceptibility of a patient, and the route of administration. The exact dose is chosen by an individual physician in view of the condition of a patient to be treated.

The present invention is directed to a method for promoting axon re-growth and behavior recovery in a subject suffering from a nerve injury. The method comprises administering a pharmaceutical composition comprising a safe and effective amount of a chondroitinase ABC (ChABC) to an injury site of the nerve injury in the subject. The subject includes a mammal, including a human. The nerve injury includes a central nervous system (CNS) injury or peripheral nervous system (PNS) injury, such as spinal cord injury. In one embodiment of the invention, a significantly improved effect on the axon re-growth and a dramatic improvement on behavior recovery after the ChABC treatment is found.

In accordance with one embodiment of the invention, the CHABC is administered to the subject by infusing the CHABC to a nerve injury site of the subject, such as through a catheter inserted into the spinal cord injury site.

According to one embodiment of the invention, the catheter may be an epidural intrathecal catheter which is inserted into the spinal cord injury site and has one end externalized for infusion of the ChABC. However, the mode of infusing the CHABC shall not be limited as such. Other methods for infusing or administering the CHABC to the subject may also be encompassed by the present invention as long as they provide a non-toxic and effective dosage to the subject.

According to embodiments of the present invention, an infusion solution comprises about 0.5 to about 10 U/ml CHABC, preferably about 0.5 U/ml to about 5 U/ml ChABC and more preferably about 1 U/mil CHABC. About 1 μl to about 100 μl of the infusion solution can be administered to a subject per infusion to provide a safe and effective dosage of CHABC of about 0.5 mU to about 1 U. The ChABC can be administered to the subject once daily, once every other day, once weekly, etc., depending on the individual need of the subject.

The present invention also relates to a pharmaceutical composition for promoting axon re-growth and behavior recovery in a subject suffering from a nerve injury. The pharmaceutical composition comprises a safe and effective amount of a chondroitinase ABC (ChABC) and a pharmaceutically acceptable carrier. Pharmaceutically acceptable carriers known to those skilled in the art can be used in the composition. Examples of such pharmaceutically acceptable carriers include, but are not limited to, sterile water and saline solution among others known to those skilled in the art in view of the present disclosure. Other active or non-active ingredients may be added without diminishing the therapeutic effect of the ChABC.

In certain embodiments of the present invention, the pharmaceutical composition comprises about 0.5 to about 10 U/ml ChABC, preferably about 0.5 U/ml to about 5 U/ml ChABC and more preferably about 1 U/ml ChABC.

It is noted that CSPGs are upregulated via astrocytes and oligodendrocytes to limit axonal regeneration. According to the invention, an infusion of ChABC to the injury site degrades the CSPGs, allowing the axonal regeneration to take place.

In accordance with an example of the invention, an epidural intrathecal catheter is inserted via an incision to a spinal cord injury site of the subject suffering from a nerve injury. The incision may then be closed, leaving one end of the epidural intrathecal catheter externalized for infusion of the ChABC. A significant enhancement effect of ChABC on the axon re-growth as well as behavior recovery of a subject suffering from a nerve injury was found in the invention.

The CHABC or the pharmaceutical composition containing ChABC may be given depending on the individual subject's need, such as once a day, or once a week in a human. In one embodiment of the invention, the subject, such as a rat, is administrated with about 5 to 10 μl of a composition comprising about 1 U/ml of ChABC every other day for two weeks (total 8 times).

The invention will now be described in further detail with reference to the following specific, non-limiting examples.

### EXAMPLE 1

### The Insertion of Catheter and Infusion of Chondroitinase ABC after T8 Complete Spinal Cord Transection

Female Sprague-Dawley (SD) rats (each weighed 250 to 300 g) were used in the experiment. All surgeries were done under inhalation anesthesia with isoflurance and above homoeothermic blanket for keeping body temperature at about 37° C. The skin over foramen magnum was prepared and a longitudinal midline incision was made to expose the magnum and C1 lamina. The epidural space was created just above the C1 lamina and the catheter was inserted to reach the T8-T9 level. The incision was closed layer by layer and the catheter was externalized for infusion of chondroitinase ABC.

One week after catheter insertion and the confirmation of normal hindlimbs movement, these animals received T8 total laminectomy to expose the spinal cord and the inserted catheter at T8 level. The complete transection of spinal cord was done by lifting up both stumps for the completion of spinal cord transection and both stumps were approximated together. The catheter was cut at the T8 cord level also for the infusion of ChABC. The first dose of ChABC was given 2, 4 and 6 weeks after the transection of spinal cord through the epidural intrathecal catheter. The incision was closed layer by layer. After surgery, the animals were immediately placed on homoeothermic blanket and kept under heating lamp for first week. Urine was discharged via catheterization twice daily until the urination function became normal. To prevent urinary tract infection, prophylactic antibiotic was administered once daily until the animal voided its own urine.

The female SD rats were divided into three groups according to the interval between spinal cord transection and infusion of ChABC. The ChABC enzyme was infused at three different concentrations, 1, 5 and 10 U/ml, after complete spinal cord transection. ChABC was given every other day for a duration of two weeks (total 8 times, each time 6 μl) under inhalation anesthesia. The externalized tube was cut and removed two weeks after the last infusion of ChABC. In the control group, the female SD rats received (1) T8 spinal cord transection (tx) only (T8 tx only), (2) T8 spinal cord transection and insertion of epidural intrathecal catheter only (T8 tx+tube only), (3) injury, catheter insertion and normal saline infusion only (T8 tx+saline) until 8 weeks after the final dose of ChABC infusion.

### EXAMPLE 2

### Behavioral Assessment

All animals received behavioral testing every week post-surgery for 8 weeks. All behavioral tests were videotaped and two examiners who participated in behavior evaluation were blinded to each group. The hindlimb locomotor's behavior of rats was evaluated by the Basso, Beattie, Bresnahan (BBB) open field locomotor's test. Each session lasted 5 minutes. The open field locomotor's activity score ranging from 0 to 21 (0 for no movement, and 21 for normal movement) was determined by observation and scoring of behaviors involving the trunk, tail and hindlimb.

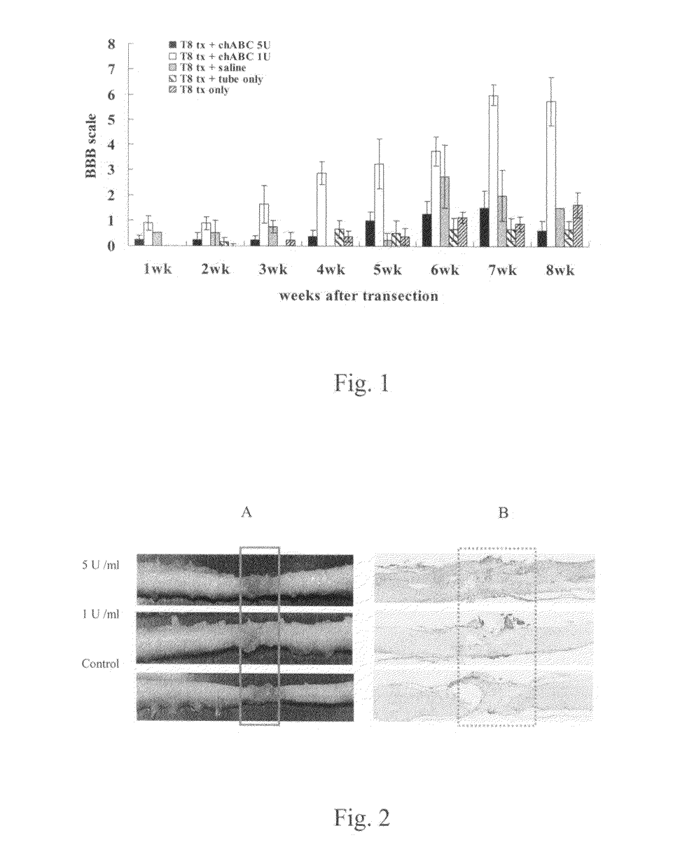

Referring to FIG. 1, there were statistical differences in the hind limbs locomotor's function evaluation between rats treated with CHABC 1 U/ml (T8 tx+ChABC 1 U), ChABC 5 U/ml group (T8 tx+ChABC 5 U) and the control groups (T8 tx+saline, T8 tx+tube only and T8 tx only), 3 weeks after surgery and CHABC treatment, and the differences persisted up to 8 weeks and longer.

### EXAMPLE 3

### Anterograde Labeling of Axons Crossing the Transection Site

Following 8 weeks post surgery, the female SD rats were anesthetized under inhalation of isoflurane. The spinal cord was exposed over the T10 level and 4% WGA-HRP (wheat germ agglutinin-horseradish peroxidase) was injected into the motor cortex of the brain via a micro-syringe. Three sites (to disperse injections on each side 0.24 μl×3) were injected by a slow pumping system. The animals were sacrificed for transcardial perfusion with 4% paraformaldehyde under anesthesia two days after microinjection. The spinal cord, together with brain stem were removed, post-fixed and cryo-preserved in 30% sucrose overnight for serial section. The spinal cord was sectioned longitudinally and the brain stem was sectioned coronary at about 30 μm thickness.

### EXAMPLE 4

### Immunohistochemistry

The spinal cords were collected, immersed in 4% paraformaldehyde in phosphate buffer overnight and then were transferred to a 30% sucrose solution. The horizontal or transverse cry-sections (each having a 20 μm thickness) of the spinal cords were placed on poly-L-lysine-coated slide for immunostaining. Following incubation with 5% bovine serum albumin in PBS for 30 min, monoclonal antibodies to CS-56 (1:500; Sigma, St. Louis, Mo.), 2B6 (1:5000; Seikagaku Corporation), GAP-43 (1:1000; Sigma, St. Louis, Mo.) and NG2 were used for primary antibodies. Appropriate secondary antibody plus avidin biotin complex (ABC) procedure was used for peroxidase staining. Negative control was verified by omission of the primary antibody to CS-56 (a type of CSPG), 2B6 (degradation products of CSPG) and GAP-43 (a marker for newly formed axon), and antibodies were also used for the evaluation of the degradation of CSPG and the regeneration of axons across the lesion site.

The morphological scar tissues were observed in rats treated with ChABC 1 U/ml and CHABC 5 U/ml by comparison with the control group as shown in FIG. 2A. However, large cysts were observed near the transection site in the ChABC 5 U/ml group as shown in FIG. 2B.

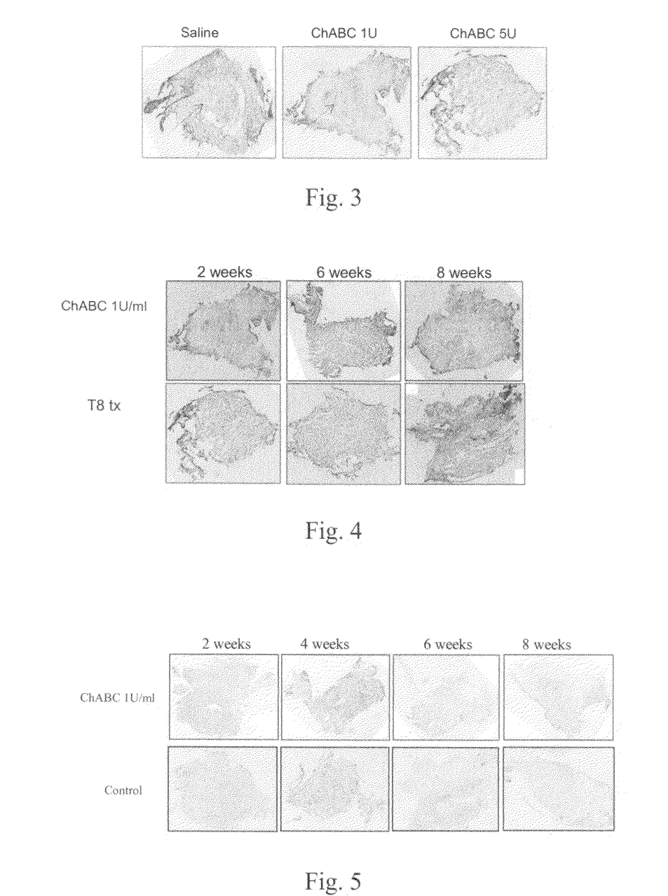

The immunostaining of CS-56, which was a complete CSPGs structure of scar over the region of spinal cord injury, revealed dramatic decrease in the group treated with ChABC 1 U/ml compared with the group treated with ChABC 5 U/ml and the control groups 8 weeks after spinal cord injury and ChABC treatment as shown in FIG. 3. The undigested scar tissues were strongly immunopositive of CS-56. The lowest expression of CS-56 within 2 weeks after 1 U/ml ChABC treatment was detected, but CS-56 still increased for 4 weeks after ChABC treatment as shown in FIG. 4. In the group treated with ChABC 1 U/ml, the CS-56 immunostaining was reached the peak level 2 weeks after spinal cord injury compared with the T8-transection group, whose CS-56 immunostaining reached peak level at 6 weeks and persisted up to 8 weeks.

The 2B6 expression in the ChABC 1 U/ml group was stronger than the control groups. There were no differences in the immunostaining of 2B6 (for degradation products of CSPGs by ChABC) between the groups treated with ChABC 1 U/ml and ChABC 5 U/ml group and the control group for 8 weeks after ChABC treatment. Referring to FIG. 5, the 2B6 immunostaining was observed in the group treated with 1 U/ml ChABC for 2 weeks after the ChABC treatment to compare with the control group.

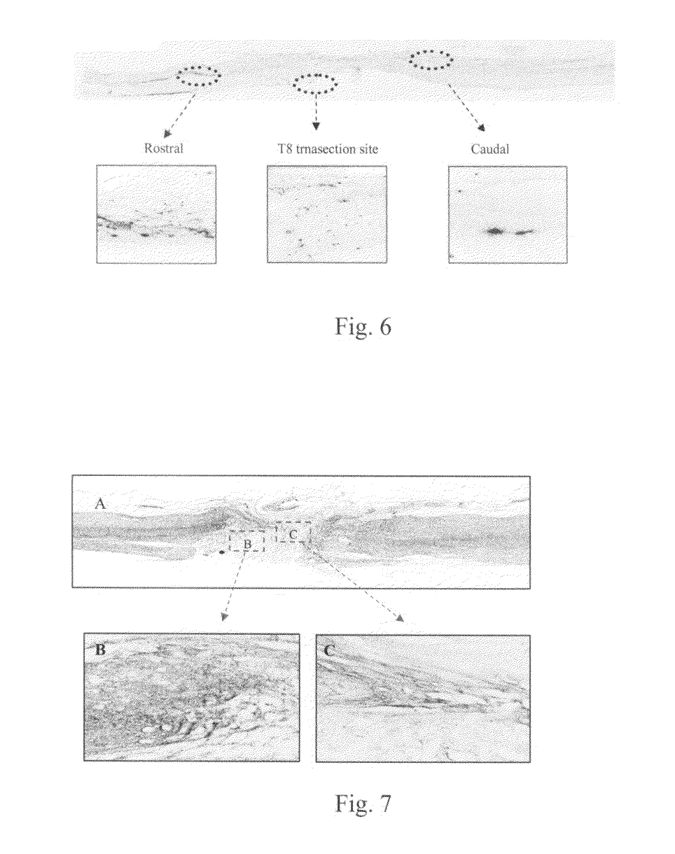

Wheat-germ agglutinated-horse radish peroxidase (WGA-HRP) labeled axons were observed across the transection site in the ChABC 1 U/ml group as shown in FIG. 6. Small size scar did not block the re-growth of axons. Referring to FIG. 7A, a few GAP-43 immunopositive processes were observed across the T8 transection site in the group treated with CHABC 1 U/ml 8 weeks after transection. In the rostral stump lesion site, a large number of axons were observed as shown in FIG. 7B. In the central area of the scar tissue, less and thinner axons were found as shown in FIG. 7C.

It should be understood by one having an ordinary level of skill in the art in view of the present disclosure that the present method is equivalently applicable to treating any vertebrates suffering from the nerve injury. The vertebrates include but not limited to humans, commercially relevant mammals such as cattle, pigs, horses, sheep, cats, dogs, mice, rats, rabbits, and pets, birds including commercially relevant birds such as chickens, ducks, geese, and turkeys.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/we claim:

1. A method for promoting axon re-growth and behavior recovery in a subject suffering from a nerve injury, the method comprising administering a pharmaceutical composition comprising a safe and effective amount of a chondroitinase ABC (CHABC) to an injury site of the nerve injury in the subject.

2. The method according to claim 1, wherein the pharmaceutical composition comprises about 0.1 U/ml to about 10 U/ml of the CHABC.

3. The method according to claim 1, wherein the pharmaceutical composition comprises about 0.5 U/ml to about 5 U/ml of the CHABC.

4. The method according to claim 1, wherein the pharmaceutical composition comprises about 1 U/ml of the CHABC.

5. The method according to claim 1, wherein the pharmaceutical composition is administered to the injury site by infusion.

6. The method according to claim 5, wherein the pharmaceutical composition is administered via an epidural intrathecal catheter.

7. The method according to claim 5, wherein about 1 μl to about 100 μl of the pharmaceutical composition is administered to the injury site per infusion.

8. The method according to claim 7, wherein the infusion is performed once daily or once every other day.

9. The method according to claim 1, wherein the nerve injury comprises a central nervous system (CNS) injury or a peripheral nervous system (PNS) injury.

10. The method according to claim 9, wherein the nerve injury comprises a spinal cord injury.

11. The method according to claim 1, wherein the subject is a mammal.

12. The method according to claim 1, wherein the subject is a human.

13. A pharmaceutical composition for promoting axon re-growth and behavior recovery in a subject suffering from a nerve injury, the pharmaceutical composition comprising a safe and effective amount of a chondroitinase ABC (ChABC) and a pharmaceutically acceptable carrier.

14. The pharmaceutical composition according to claim 13 comprising about 0.1 U/ml to about 10 U/ml of the ChABC.

15. The pharmaceutical composition according to claim 13 comprising about 0.5 U/ml to about 5 U/ml of the ChABC.

16. The pharmaceutical composition according to claim 13 comprising about 1 U/ml of the ChABC.
